(19) United States
(12) Patent Application Publication
(10) Pub. No.: US 2022/0142810 A1
(43) Pub. Date: May 12, 2022

(54) Female Urinary Catheter System

(71) Applicant: Garrett Whittaker, Torrington, CT (US)

(72) Inventor: Garrett Whittaker, Torrington, CT (US)

(21) Appl. No.: 17/525,854

(22) Filed: Nov. 12, 2021

Related U.S. Application Data
(60) Provisional application No. 63/112,775, filed on Nov. 12, 2020.

(51) Int. Cl. A61F 5/455; A61F 5/44; A61L 2/10

(52) U.S. Cl. CPC A61F 5/4556; A61F 5/4408; A61L 2202/24; A61L 2/10; A61L 2202/11; A61F 5/4553

(57) ABSTRACT

A female urinary catheter system may be provided comprising a drainage aperture element with an outer edge surrounding an inner aperture. The system may utilize a catheter conduit coupled to the inner aperture and may comprise one or more conduit segments which each have a first terminal end and a second terminal end. Further, the system may utilize a storage receptacle coupled to the catheter conduit with the storage receptacle comprising a leg coupling mechanism. Additionally, an anchor element may be coupled adjacent the drainage aperture element to provide positional stability to the drainage aperture element

### CROSS-REFERENCE TO RELATED APPLICATIONS

This application claims priority to U.S. Provisional Patent Application Ser. No. 63/112,775 entitled “Female Urinary Catheter System” filed Nov. 12, 2020. The contents of this application are hereby incorporated by reference in their entirety.

### BACKGROUND OF THE INVENTION

Urinary catheters are one sector within a larger domestic catheter market that includes cardiovascular catheters, intravenous catheters, neurovascular catheters and the like among other specialty catheters. Within the United States, the larger catheter market is estimated at a value of over $10 billion USD with urinary or urology catheters comprising roughly 20% of that value. Typical urinary catheters are hollow, partially flexible tubes that collect urine from the bladder and lead to one or more drainage bags or other portable or stationary receptacles. Urinary catheters may come in many sizes and types and can be made of rubber, PVC plastic, silicone and the like. Typical types of urinary catheters include those that are inserted into the user's urethral passage to allow for urine to flow therethrough while securely being retained in place by the frictional engagement between the user's urethral passage and the catheter conduit.

Catheters are generally only necessary when a patient can't control the emptying of their bladder. In the case where they cannot empty their bladder, urine will build up and generate pressure in the user's kidneys. Such pressure can lead to failure of kidney function which can result in irreversible damage to the kidneys and may require costly and undesirable chronic dialysis treatments. Most catheters are only necessary until the user regains their ability to urinate on their own which is typically a short period of time. Elderly users and those with a permanent injury, disability or other severe illness may require use of a urinary catheter for a much longer time or even permanently in some cases.

A user may be directed by a doctor to begin using a catheter if they cannot control when they urinate, have urinary incontinence, have urinary retention and the like. Some users may not be able to urinate on their own for reasons including blocked urine flow due to bladder or kidney stones, blood clots in the urine, severe enlargement of the prostate gland, surgery on the prostate gland, surgery in the genital area such as a hip fracture repair or hysterectomy, injury to the nerves of the bladder, a spinal cord injury, a condition that impairs the user's mental function such as dementia, consumption of medications that impair the user's ability for the bladder muscles to squeeze which may cause urine to remain stuck in the bladder and the like, among other reasons.

The typically utilized types of catheters include indwelling catheters, external catheters, and short-term catheters. Indwelling catheters may include urethral catheters and suprapubic catheters. An indwelling catheter is a catheter that resides in the bladder, such as a Foley catheter. This type of catheter may be used for both short and long durations of use. In practice, a nurse typically inserts an indwelling urethral catheter into the user's bladder through the urethra. Alternatively, a healthcare provider may insert the catheter into the bladder through a tiny hole in the abdomen which is referred to as a suprapubic catheter. In the suprapubic case, a tiny balloon at the end of the catheter is inflated with water to prevent the tube from sliding out of the body. The balloon can then deflate when the catheter needs to be removed.

External catheters such as condom catheters are catheters placed outside the body which are typically necessary for male users who, while not having urinary retention problems, have serious functional or mental disabilities such as dementia. In some instances, a device that looks like a condom covers the penis head while a conduit leads from the condom device to a drainage bag. External catheters are generally more comfortable and carry a lower risk of infection than indwelling catheters but they usually need to be changed daily with some models being designed for longer chronic use. Long-term external catheters can cause less skin irritation than those requiring daily removal and reapplication.

In some cases, a short-term catheter, or intermittent catheter, may be desired for a user who may only need a catheter for a short period of time, such as after surgery until the bladder empties. After the bladder empties, it's necessary to remove the short-term catheter and so many healthcare providers refer to this type of catheter as an in-and-out catheter. In the home setting use of intermittent catheters, users may be trained to apply the catheter themselves or with the help of a caregiver. Application can be done through the urethra or through the hole created in the lower abdomen for catheterization.

A catheter-associated urinary tract infection (CAUTI) is known as one of the most common infections a person can contract during a stay in the hospital. Indwelling catheters are the cause of almost all cases of this type of infection. Most commonly, bacteria or fungi may enter the user's urinary tract via the catheter where they may multiply thus causing an infection. However, there are a number of other ways infection can occur during catheterization including the catheter becoming contaminated upon insertion, the drainage bag not being emptied often enough, bacteria from a bowel movement contaminating the catheter, urine in the catheter bag back flowing back into the bladder, the catheter not being cleaned regularly enough and the like.

A CAUTI may exhibit similar symptoms to a typical urinary tract infection (UTI) which include cloudy urine, blood in the urine, strong urine odor, urine leakage around the catheter, pain or discomfort in the lower back or stomach, chills, fever, unexplained fatigue, vomiting and the like. CAUTIs can be difficult to diagnose in if the user is already hospitalized because similar symptoms may be part of the originally diagnosed illness for which hospitalization was required. In the elderly, changes in mental status or confusion can be additional signs of a CAUTI being present.

Upon diagnosis of a CAUTI, prompt treatment is essential as an untreated UTI in general can lead to more serious kidney infections. Additionally, catheter users likely already have preexisting conditions that compromise their immune systems and may have required their use of a catheter in the first place. Moreover, utilizing bodily resources to fight off a CAUTI can cause further immune system stress upon the user which makes the user more vulnerable to additional future infections.

CAUTIs tend to be more resistant to treatment than other UTIs, a common feature in general for hospital-acquired infections. Further, a doctor may prescribe antibiotics to kill off any potentially harmful bacteria causing the infection which places the user at further risk of developing resistance to the given antibiotics. If the infection causes bladder spasms, the doctor may further prescribe an anti-spasmodic to lessen bladder pain.

Generally, indwelling catheters are more risky to the user than externally-applied catheters and so their use would be discouraged relative to that of an externally-applied catheter if all else were equal. However, there do not exist solutions for externally-applied catheters for female users that satisfy the needs of the market and so indwelling catheters are still more commonly used which places the health burden of infectious risk upon the patient along with the associated medical cost burden for treating such infections.

Therefore, it would be advantageous to provide an external catheter that serves the needs of female users. However, common issues with external catheters for females include the poor mechanical coupling of the external catheter to the female users's urinary anatomy. Specifically, a male user's urinary anatomy provides a larger surface area of external structure upon which to secure the external catheter. A female user's urinary anatomy provides significantly less surface area of external structure for the same purpose and so the rate of decoupling of the external catheter from the female's urinary anatomy is significantly higher than that in the case of male user's external catheters.

Consequently, it would be advantageous to provide a female urinary catheter system that provides a female user with a lower rate of decoupling between the external catheter and the urinary anatomy. It would be further advantageous to provide a female user with such a solution that is also portable in nature for those female users who are capable and willing to lead a more active life than those patients who are bedridden. Moreover, it would be advantageous or provide a female urinary catheter system that allows a female user to customize the length and fit of the external catheter to more precisely fit the unique needs of the given female user's body dimensions and urinary anatomy dimensions. Additionally, it would be advantageous to provide a female urinary catheter system that is fabricated from one or more materials that are selected to have one or more of anti-pathogenic properties, antimicrobial properties, anti-fungal properties, antiviral properties, antibacterial properties and the like or any combination thereof.

### BRIEF DESCRIPTION OF THE DRAWINGS

FIG. 1 is an overview of a female urinary catheter system in accordance with some embodiments of the present invention.

FIG. 2 is an overview of a female urinary catheter system in use by a female user coupled to the female user's leg in accordance with some embodiments of the present invention.

FIG. 3 is an overview of a female urinary catheter system utilizing a portable pump element operably coupled thereto in accordance with some embodiments of the present invention.

FIG. 4 is an exploded view of a drainage aperture element decoupled from a catheter conduit of a female urinary catheter system in accordance with some embodiments of the present invention.

FIG. 5A is a detailed view of an anchor element with a deformable anchor disposed within a female user's uterus of a female urinary catheter system in accordance with some embodiments of the present invention.

FIG. 5B is a detailed view of an anchor element with a t-frame anchor disposed within a female user's uterus of a female urinary catheter system in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a female urinary catheter system” also includes a plurality of female urinary catheter systems and the like.

In some embodiments, a female urinary catheter system is provided comprising a drainage aperture element comprising an outer edge surrounding an inner aperture; a catheter conduit coupled to the inner aperture, wherein: the catheter conduit comprises one or more conduit segments each comprising a first terminal end and a second terminal end; and a storage receptacle coupled to the catheter conduit, wherein: the storage receptacle comprises a leg coupling mechanism.

In some embodiments, an anchor element may be coupled adjacent the drainage aperture element.

In some embodiments, the anchor element comprises a vaginal deformable anchor shaped to be secured within a vaginal canal.

In some embodiments, the anchor element comprises a uterine deformable anchor shaped to be secured within a cervical canal.

In some embodiments, the anchor element comprises a uterine t-frame anchor shaped to be secured within a uterine canal.

In some embodiments, the anchor element comprises a support post coupled adjacent the drainage aperture element.

In some embodiments, the anchor element comprises a T-joint conduit coupling element coupled to the catheter conduit adjacent the drainage aperture element.

In some embodiments, one or more of the drainage aperture element, the catheter conduit, the storage receptacle and the anchor element comprises one or more materials having anti-pathogenic properties, antimicrobial properties, anti-fungal properties, antiviral properties, antibacterial properties or any combination thereof.

In some embodiments, one or more of the drainage aperture element, the catheter conduit, the storage receptacle and the anchor element comprises one or more of chemical structures comprising iodine, chemical structures comprising silver, chemical structures comprising gold, chemical structures comprising copper, chemical structures comprising cobalt, chemical structures comprising zinc, chemical structures comprising graphene, chemical structures comprising curcumin or any combination thereof.

In some embodiments, one or more of the drainage aperture element, the catheter conduit, the storage receptacle and the anchor element comprises silicone, PVC, polyethylene, polycarbonate, PEEK, polyetherimide, PEI, polypropylene, polysulfone, polyurethane or any combination thereof.

In some embodiments, the first terminal end of each conduit segment is disposed towards the drainage aperture element and the second terminal end of each conduit segment is disposed towards the storage receptacle.

In some embodiments, when a first conduit segment is coupled to a second conduit segment, a second terminal end of the first conduit segment is overlapped by a first terminal end of the second conduit segment.

In some embodiments, one or more of the drainage aperture element, the catheter conduit and the storage receptacle comprises one or more illumination elements.

In some embodiments, the one or more illumination elements emit light within the wavelength range of 200 to 450 nanometers.

In some embodiments, the one or more illumination elements comprise one or more LEDs.

In some embodiments, the one or more illumination elements comprise one or more luminescing materials.

In some embodiments, a pump element may be disposed between the drainage aperture element and the storage receptacle.

In some embodiments, one or more of the drainage aperture element and the catheter conduit comprises a bodily coupling mechanism for coupling around a human body part.

In some embodiments, a female urinary catheter system is provided comprising a drainage aperture element coupled to a catheter conduit, wherein: the catheter conduit comprises one or more conduit segments each comprising a first terminal end and a second terminal end, and one or more of the drainage aperture element and the catheter conduit comprises a bodily coupling mechanism for coupling around a human body part; an anchor element coupled adjacent the drainage aperture element, wherein: the anchor element comprises a deformable anchor shaped to be secured within a vaginal canal or a cervical canal; and a storage receptacle coupled to the catheter conduit, wherein: the storage receptacle comprises a leg coupling mechanism, and wherein one or more of the drainage aperture element, the catheter conduit, the storage receptacle and the anchor element comprises one or more materials having anti-pathogenic properties, antimicrobial properties, anti-fungal properties, antiviral properties, antibacterial properties or any combination thereof.

In some embodiments, a female urinary catheter system is provided comprising a drainage aperture element coupled to a catheter conduit, wherein: the catheter conduit comprises one or more conduit segments each comprising a first terminal end and a second terminal end, and one or more of the drainage aperture element and the catheter conduit comprises a bodily coupling mechanism for coupling around a human body part; an anchor element coupled adjacent the drainage aperture element, wherein: the anchor element comprises a deformable anchor shaped to be secured within a vaginal canal or a cervical canal; a storage receptacle coupled to the catheter conduit, wherein: the storage receptacle comprises a leg coupling mechanism, and wherein one or more of the drainage aperture element, the catheter conduit, the storage receptacle and the anchor element comprises one or more of chemical structures comprising iodine, chemical structures comprising silver, chemical structures comprising gold, chemical structures comprising copper, chemical structures comprising cobalt, chemical structures comprising zinc, chemical structures comprising graphene, chemical structures comprising curcumin or any combination thereof; and a pump element disposed between the drainage aperture element and the storage receptacle.

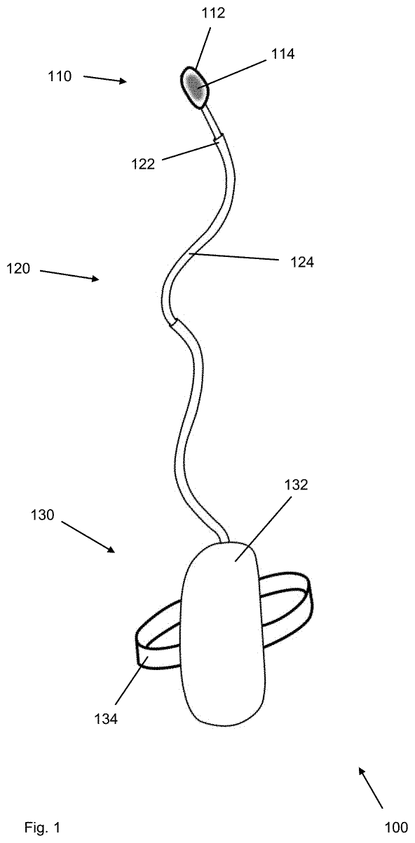

Exemplary embodiments of the present invention are illustrated in the accompanying figures. As shown in FIG. 1, an overview of a female urinary catheter system 100 is provided in accordance with some embodiments of the present invention. The female urinary catheter system 100 may comprise a drainage aperture element 110, a catheter conduit 120 and a storage receptacle 130.

The drainage aperture element 110 may comprise an outer edge 112 and an inner aperture 114 disposed within the outer edge 112. As shown in FIG. 1, the outer edge 112 may comprise an oval shape that encircles a similarly shaped inner aperture 114. However, while the outer edge 112 is generally disposed within the same plane when not deformed, the inner aperture 114 may comprise a concave shape relative its operable coupling to the outer edge 112. The concave shape may provide advantageous liquid drainage properties such that any expelled urine that is incident upon the inner surface of the inner aperture 114 may drain towards a central port of the inner aperture 114 that fluidically couples to the catheter conduit 120. In some embodiments, the concave shape may comprise a substantially parabolic shape that causes incident urinary liquid to be directed toward the central port of the inner aperture 114 and down into the catheter conduit 120.

The drainage aperture element 110 may take any suitable shape that conforms to the external urinary anatomy of a female user, but is preferably a flat flexible conformable ring shape or a flat flexible conformable oval shape in order to more easily conform to the external surface of the female user's urinary anatomy. In some embodiments, the outer edge 112 of the drainage aperture element 110 may be customizable and deformable in nature to allow the female user to manipulate the shape of the outer edge 112 to better meet the unique contour of the female user's urinary anatomy. For instance, the outer edge 112 may comprise a thin-gauge gooseneck-type material.

While the inner aperture 114 is preferably concave shaped and more preferably substantially parabolically shaped, the inner aperture 114 may also be flexibly conformable and deformable in nature to allow for a more comfortable fit when worn under tight-fitting clothing. Additionally, such a flexibly conformable and deformable nature would make the female urinary catheter system 100 less noticeable to third-party observers when worn by the female user out in public under clothing such as underwear, pants, dresses and the like.

Additionally, the outer edge 112 of the drainage aperture element 110 may comprise an adhesive disposed between the skin around the urinary anatomy of the female user and the external surface of the outer edge 112. Specifically, the outer edge 112 may comprise the adhesive disposed thereupon prior to application over the female user's urinary anatomy. In other embodiments, the outer edge 112 and/or the inner aperture 114 may comprise a retention band to be worn around the female user's waist and/or legs. In further embodiments, no adhesive or retention band may be utilized, but rather the drainage aperture element 110 may be secured in place via the underwear of the female user being worn thereover.

The catheter conduit 120 may comprise a terminal coupling joint 122 disposed at each end of one or more conduit segments 124 utilized in the catheter conduit 120. The terminal coupling joints 122 may be disposed in frictional coupling engagement to another of the terminal coupling joints 122 of another conduit segment 124. Each of the one or more conduit segments 124 may be modularly coupled to one another and removably coupled to one another. In some embodiments, the conduit segments 124 that are disposed closer to the drainage aperture element 110 may be coupled to an adjacent conduit segment 124 farther from the drainage aperture element 110 in a manner such that the farther conduit segment 124 overlaps the closer conduit segment 124 at the location of the associated terminal coupling joints 122. Such a coupling configuration allows for urinary fluid to flow from the drainage aperture element 110 through the catheter conduit 120 to the storage receptacle 130 without any of the urinary fluid getting caught in the catheter conduit 120 or leaking therefrom.

Additionally, the catheter conduit 120 may be fabricated from one or more non-toxic and biocompatible materials. In some embodiments, the non-toxic and biocompatible materials may include, but are not limited to, silicone, foam or other like materials exhibiting soft, deformable, pliable and durable materials such as related polymer materials. Other non-toxic and biocompatible including PVC, polyethylene, polycarbonate, PEEK, polyetherimide, PEI, polypropylene, polysulfone, polyurethane and the like.

Further, the drainage aperture element 110, the catheter conduit 120 and the storage receptacle 130 may be coated with one or more materials having anti-pathogenic properties, antimicrobial properties, anti-fungal properties, antiviral properties and/or antibacterial properties. Such materials may include, but are not limited to, compounds comprising iodine and derivative chemical structures and ions, nano-silver and derivative chemical structures and ions, nano-gold and derivative chemical structures and ions, nano-copper and derivative chemical structures and ions, nano-cobalt and derivative chemical structures and ions, nano-zinc and derivative chemical structures and ions, graphene based compounds, curcumin based compounds and the like or any combinations thereof.

Moreover, the drainage aperture element 110, the catheter conduit 120 and the storage receptacle 130 may comprise one or more illumination elements having anti-pathogenic properties, antimicrobial properties, anti-fungal properties, antiviral properties and/or antibacterial properties. For instance, the one or more illumination elements may comprise LEDs, luminescing materials and coatings and the like or any combination thereof. The one or more illumination elements may emit light within the UV or near-UV spectrum between wavelength ranges including the 200-450 nm band of light.

The catheter conduit 120 may be operably coupled to the storage receptacle 130 to allow transport of urinary fluid thereto. In some embodiments, the catheter conduit 120 may be situated in a position that is vertically above the storage receptacle 130 such that gravity enables transport of the urinary fluid into the storage receptacle 130. In other embodiments, a suction element or fluid pump element may be utilized to generate a low-level vacuum that allows for urinary fluid to be transported from the drainage aperture element 110 to the storage receptacle 130 even when the drainage aperture element 110 is disposed within a vertical plane that is vertically lower than the storage receptacle. Use of the low-level suction would be advantageous even in instances when the drainage aperture element 110 will always be disposed above storage receptacle 130 since the low-level vacuum will aid in the gravity-powered flow of the urinary fluid to comprehensively flow into the storage receptacle 130 and prevent back flow of the urinary fluid back into the female users's bladder which is known to cause urinary tract infections or more serious ailments such as kidney infection or even failure.

The storage receptacle 130 may comprise a urinary fluid reservoir 132 and a coupling mechanism 134. The urinary fluid reservoir 132 may be shaped in any suitable manner but preferably is shaped to contain at least two full bladder movements of the average sized bladder or about 32 ounces. The urinary fluid reservoir may be fabricated from any suitable flexible material that is at least semi-transparent to incident light so as to allow disinfection from external UV or near-UV light emitted from the one or more illumination elements. The coupling mechanism 134 may take any suitable form including, but not limited to, a strap, a buckle, a clasp, hook and loop fasteners, buttons, magnets and the like or any combination thereof.

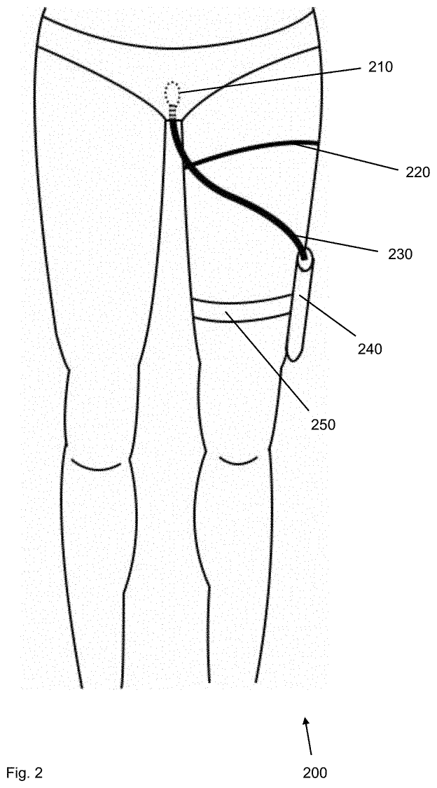

As shown in FIG. 2, an overview of a female urinary catheter system 200 in use by a female user coupled to the female user's leg is provided in accordance with some embodiments of the present invention. The female urinary catheter system 200 may comprise a drainage aperture element 210, a conduit coupling mechanism 220, a catheter conduit 230, a storage receptacle 240 and a coupling mechanism 250. The female urinary catheter system 200 of FIG. 2 may be substantially similar to that of FIG. 1 but for FIG. 2 illustrating the use case of the catheter system 200 in a portable configuration operably coupled to the female user's leg.

It would be advantageous for the female urinary catheter system 200 to comprise a portable configuration in the event the female user is not bedridden and wishes to lead a more active lifestyle. In order to place the catheter system 200 into the portable configuration, the conduit coupling mechanism 220 may be introduced into the system 200 in order to more securely couple the catheter conduit 230 to the female user's body. Without such an additional coupling mechanism, the catheter conduit 230 may move around under the female user's leg while walking around which is not desirable.

Additionally, FIG. 2 illustrates the drainage aperture element 210 being disposed under the female user's underwear and over the female user's urinary anatomy. By securing the catheter conduit 230 to the female user's upper leg, securing the storage receptacle 240 to the female user's lower leg and securing the drainage aperture element 210 under the user's underwear but over the female user's urinary anatomy, the catheter system 200 may remain in a functional configuration as a catheter even while the female user is actively leading their life doing such activities as walking and the like.

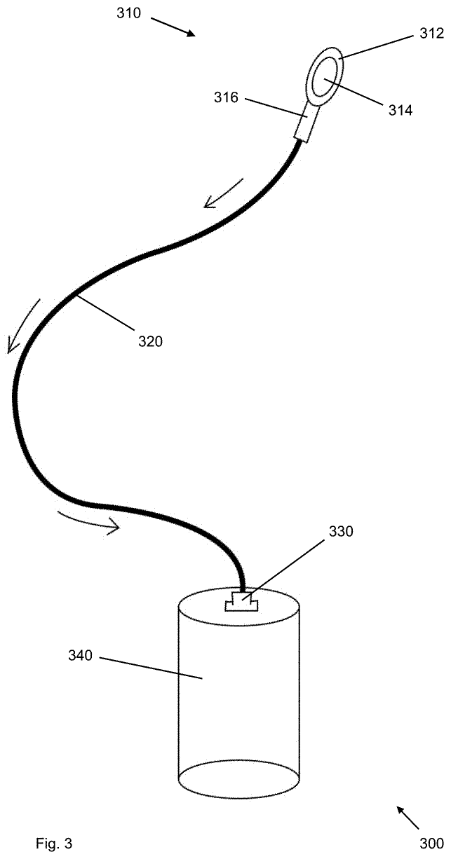

As shown in FIG. 3, an overview of a female urinary catheter system 300 utilizing a portable pump element 330 operably coupled thereto is provided in accordance with some embodiments of the present invention. The female urinary catheter system 300 may comprise a drainage aperture element 310, a catheter conduit 320, a portable pump element 330 and a storage receptacle 340.

The drainage aperture element 310 may comprise an outer edge 312, an inner aperture 314 and a coupling joint 316. The coupling joint 316 may removably and modularly couple the outer edge 312 and inner aperture 314 to the catheter conduit 320. The portable pump element 330 may be disposed along any portion of the catheter conduit 320 in order to generate a low-level vacuum therein. Preferably, the portable pump element 330 is disposed at a position adjacent the storage receptacle 340 in order to more easily generate a one-way gravity-assisted flow from the drainage aperture element 310 to the storage receptacle 340. The portable pump element 330 may comprise a wireless energy source such as a rechargeable battery but may also operate via grid power out of a standard wall outlet. The low-level suction of the portable pump element 330 may draw a corresponding low level of power from the power source.

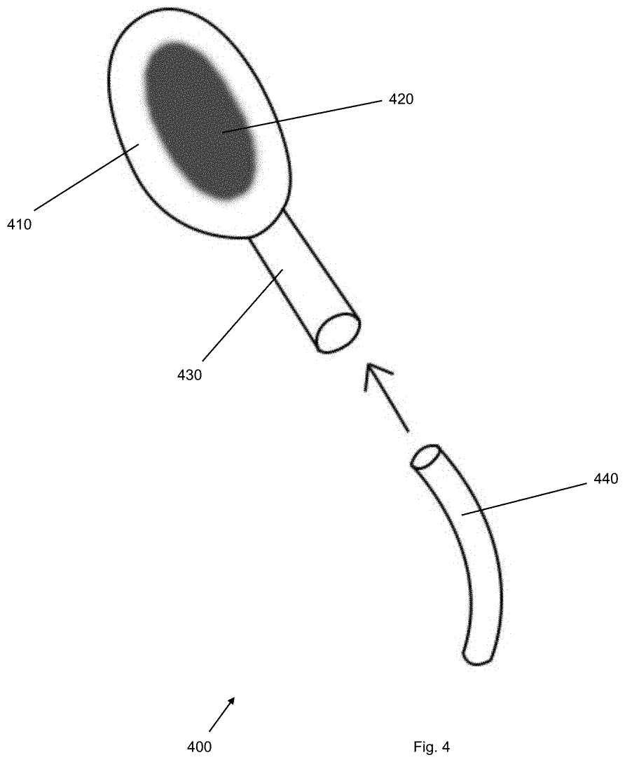

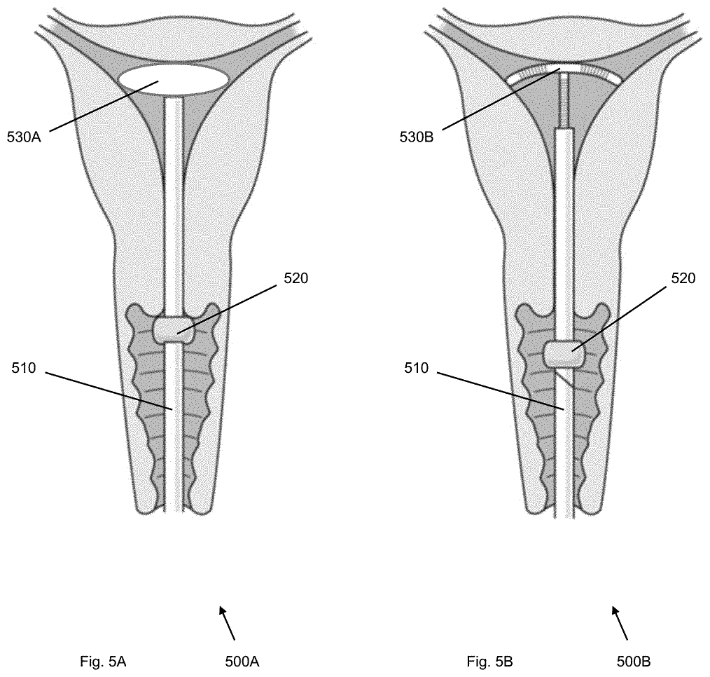

As shown in FIG. 4, an exploded view of a drainage aperture element 410 decoupled from a catheter conduit 420 of a female urinary catheter system 400 in accordance with some embodiments of the present invention. Advantageously, the drainage aperture element 410 may comprise a coupling joint 430 that may allow the drainage aperture element 410 to be modularly and removably coupled to one or more of the catheter conduit segments 440. Alternatively, one or more other elements, such as an anchor element described in FIG. 5, may be coupled between the coupling joint 430 and the catheter conduit segment 440. The use of modular components allows the catheter system 400 to be selectively constructed and adjusted by the female user to accommodate various shapes and sizes of female users.

As shown in FIG. 5A, a detailed view of an anchor element 500A with a deformable anchor 530A disposed within a female user's uterine or cervical canal of a female urinary catheter system is provided in accordance with some embodiments of the present invention. The anchor element 500A may be disposed between the coupling joint 430 and catheter conduit segment 440 of FIG. 4 by coupling therebetween a T-joint conduit coupling element (not shown). Further, a support post 510 of the anchor element 500A may be directly coupled to the T-joint conduit coupling and may be used to insert the deformable anchor 530A into the female user's vagina. The T-joint conduit coupling element may comprise a hollow conduit to allow urinary fluid to pass from the coupling joint 430 to the catheter conduit segment 440 but does not allow urinary fluid to pass through the interior of the support post 510 as the support post 510 is not a hollow conduit. This prevents back flow of urinary fluid into the vaginal canal and uterus which would increase the chance of infection. An actuation element 520 may be utilized by the female user to further grip the anchor element 500A and also deploy the deformable anchor 530A into the female user's uterus. In some embodiments, the deployment may include actuating the deformable anchor 530A from a first position within a hollow conduit at the distal end of the support post 510 to a second position disposed external from but coupled to the distal end of the support post 510 and then iteratively returning to either of the first or second positions as desired. In use, the deformable anchor 530A may be used to keep the drainage aperture element in place over the female user's urinary anatomy.

While FIG. 5A illustrates the anchor element 500A being anchored in the uterus, it is understood that the deformable anchor 530A may not be disposed within the female user's uterus at all but rather may be secured against the female user's vaginal canal walls while still achieving the same anchoring function for the female urinary catheter system. In such a vaginal wall configuration, the support post 510 may comprise a shorter length to accommodate the deformable anchor 530A being disposed closer to the T-joint conduit coupling element.

As shown in FIG. 5B, a detailed view of an anchor element 500B with a t-frame anchor 530B disposed within a female user's uterine canal of a female urinary catheter system is provided in accordance with some embodiments of the present invention. The anchor element 500B may be disposed between the coupling joint 430 and catheter conduit segment 440 of FIG. 4 by coupling therebetween a T-joint conduit coupling element. Further, a support post 510 of the anchor element 500A may be directly coupled to the T-joint conduit coupling and may be used to insert the t-frame anchor 530B into the female user's vagina. An actuation element 520 may be utilized by the female user to further grip the anchor element 500B and also deploy the t-frame anchor 530B into the female user's uterus. In use, the t-frame anchor 530B may be used to keep the drainage aperture element in place over the female user's urinary anatomy. While FIG. 5B illustrates the anchor element 500B being anchored in the uterus, it is understood that the t-frame anchor 530B may not be disposed within the female user's uterus at all but rather may be secured against the female user's vaginal canal walls while still achieving the same anchoring function for the female urinary catheter system.

With reference to FIGS. 1-5B, the drainage aperture element of the female urinary catheter system may be utilized as an external catheter in conjunction with an indwelling catheter and/or a suprapubic catheter. Additionally, the uterine anchor element or the vaginal anchor element may be utilized whether or not the drainage aperture element is being utilized complimentary with the indwelling catheter and/or the suprapubic catheter. Any of the aforementioned configurations may be utilized in modular combination with one another as desired by the female user or associated healthcare provider. For instance, a uterine anchor element or a vaginal anchor element may be utilized to anchor the position of the drainage aperture element of an external catheter and/or an indwelling catheter to ensure the functionality of the female urinary catheter system remains robust in the face of most types of movements by the female user.

With reference to FIGS. 1-5B, the drainage aperture element may not comprise an outer edge at all but rather may only comprise the inner aperture surrounded by flexible hydrophobic material having an adhesive material disposed along edges thereof for attachment to the urinary anatomy of the female user. Such a configuration would allow for use of disposable one-time-use flexible hydrophobic material elements (such as those utilized in dental dam female condoms) that may be coupled to the coupling joint or catheter conduit of the female urinary catheter system.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A female urinary catheter system, comprising:
a drainage aperture element comprising an outer edge surrounding an inner aperture;
a catheter conduit coupled to the inner aperture, wherein:
the catheter conduit comprises one or more conduit segments each comprising a first terminal end and a second terminal end; and
a storage receptacle coupled to the catheter conduit, wherein:
the storage receptacle comprises a leg coupling mechanism.

2. The female urinary catheter system of claim 1, further comprising:
an anchor element coupled adjacent the drainage aperture element.

3. The female urinary catheter system of claim 2, wherein the anchor element comprises a vaginal deformable anchor shaped to be secured within a vaginal canal.

4. The female urinary catheter system of claim 2, wherein the anchor element comprises a uterine deformable anchor shaped to be secured within a cervical canal.

5. The female urinary catheter system of claim 2, wherein the anchor element comprises a uterine t-frame anchor shaped to be secured within a uterine canal.

6. The female urinary catheter system of claim 2, wherein the anchor element comprises a support post coupled adjacent the drainage aperture element.

7. The female urinary catheter system of claim 2, wherein the anchor element comprises a T-joint conduit coupling element coupled to the catheter conduit adjacent the drainage aperture element.

8. The female urinary catheter system of claim 2, wherein one or more of the drainage aperture element, the catheter conduit, the storage receptacle and the anchor element comprises one or more materials having anti-pathogenic properties, antimicrobial properties, anti-fungal properties, antiviral properties, antibacterial properties or any combination thereof.

9. The female urinary catheter system of claim 2, wherein one or more of the drainage aperture element, the catheter conduit, the storage receptacle and the anchor element comprises one or more of chemical structures comprising iodine, chemical structures comprising silver, chemical structures comprising gold, chemical structures comprising copper, chemical structures comprising cobalt, chemical structures comprising zinc, chemical structures comprising graphene, chemical structures comprising curcumin or any combination thereof.

10. The female urinary catheter system of claim 2, wherein one or more of the drainage aperture element, the catheter conduit, the storage receptacle and the anchor element comprises silicone, PVC, polyethylene, polycarbonate, PEEK, polyetherimide, PEI, polypropylene, polysulfone, polyurethane or any combination thereof.

11. The female urinary catheter system of claim 1, wherein the first terminal end of each conduit segment is disposed towards the drainage aperture element and the second terminal end of each conduit segment is disposed towards the storage receptacle.

12. The female urinary catheter system of claim 11, wherein, when a first conduit segment is coupled to a second conduit segment, a second terminal end of the first conduit segment is overlapped by a first terminal end of the second conduit segment.

13. The female urinary catheter system of claim 1, wherein one or more of the drainage aperture element, the catheter conduit and the storage receptacle comprises one or more illumination elements.

14. The female urinary catheter system of claim 13, wherein the one or more illumination elements emit light within the wavelength range of 200 to 450 nanometers.

15. The female urinary catheter system of claim 14, wherein the one or more illumination elements comprise one or more LEDs.

16. The female urinary catheter system of claim 14, wherein the one or more illumination elements comprise one or more luminescing materials.

17. The female urinary catheter system of claim 1, further comprising:
a pump element disposed between the drainage aperture element and the storage receptacle.

18. The female urinary catheter system of claim 1, wherein one or more of the drainage aperture element and the catheter conduit comprises a bodily coupling mechanism for coupling around a human body part.

19. A female urinary catheter system, comprising:
a drainage aperture element coupled to a catheter conduit, wherein:
the catheter conduit comprises one or more conduit segments each comprising a first terminal end and a second terminal end, and one or more of the drainage aperture element and the catheter conduit comprises a bodily coupling mechanism for coupling around a human body part;
an anchor element coupled adjacent the drainage aperture element, wherein:
the anchor element comprises a deformable anchor shaped to be secured within a vaginal canal or a cervical canal; and
a storage receptacle coupled to the catheter conduit, wherein:
the storage receptacle comprises a leg coupling mechanism, and wherein one or more of the drainage aperture element, the catheter conduit, the storage receptacle and the anchor element comprises one or more materials having anti-pathogenic properties, antimicrobial properties, anti-fungal properties, antiviral properties, antibacterial properties or any combination thereof.

20. A female urinary catheter system, comprising:
a drainage aperture element coupled to a catheter conduit, wherein:
the catheter conduit comprises one or more conduit segments each comprising a first terminal end and a second terminal end, and one or more of the drainage aperture element and the catheter conduit comprises a bodily coupling mechanism for coupling around a human body part;
an anchor element coupled adjacent the drainage aperture element, wherein:
the anchor element comprises a deformable anchor shaped to be secured within a vaginal canal or a cervical canal;
a storage receptacle coupled to the catheter conduit, wherein:
the storage receptacle comprises a leg coupling mechanism, and wherein one or more of the drainage aperture element, the catheter conduit, the storage receptacle and the anchor element comprises one or more of chemical structures comprising iodine, chemical structures comprising silver, chemical structures comprising gold, chemical structures comprising copper, chemical structures comprising cobalt, chemical structures comprising zinc, chemical structures comprising graphene, chemical structures comprising curcumin or any combination thereof; and
a pump element disposed between the drainage aperture element and the storage receptacle.
